(19) United States
(12) Patent Application Publication
(10) Pub. No.: US 2017/0206996 A1
(43) Pub. Date: Jul. 20, 2017

(54) PRACTICAL METHOD FOR FABRICATING FOAM INTERSPACED ANTI-SCATTER GRID AND IMPROVED GRIDS

(71) Applicant: Turtle Bay Partners,LLC, Littlestown, PA (US)

(72) Inventor: Thomas J. Beck, Catonsville, MD (US)

(21) Appl. No.: 15/327,909

(22) PCT Filed: Jul. 23, 2015
(86) PCT No.: PCT/US2015/041749

§ 371 Date: Jan. 20, 2017

Related U.S. Application Data
(60) Provisional application No. 62/028,161, filed on Jul. 23, 2014.

(51) Int. Cl. G21K 1/10

(52) U.S. Cl. CPC G21K 1/10; A61B 6/4291

(57) ABSTRACT

A device for, and method of manufacture of, a focused and-scatter grid for improving the image contrast of x-ray images produced in medical, veterinary or industrial applications. The grid comprising a series of modular units so juxtaposed with each other as to form a series of focused channels for the passage of the focused imaging x-rays. The modules comprise a series of focusing strips of a heavy metal or a series of mating solid arcuate forms, formed of a polymer and having on at least one side surface a layer of heavy metal.

### FIELD OF THE INVENTION

The present invention generally relates to grids used in radiation imaging including x-ray imaging.

### BACKGROUND OF THE INVENTION

The fields of medical and industrial radiography use the technique of directing beams of electromagnetic radiation toward an object (or part of the human body), so that the radiation passes through the object, to obtain an image of the interior of the object, that is otherwise difficult to access or view directly without cutting through the body or other object. Usually, the electromagnetic radiations used for imaging purposes are x-rays, which tend to scatter as they travel through the object to be imaged.

The scattered x-rays contribute to the degradation of the image of the object and more particularly to the degradation of the image contrast. The x-rays that travel through the object that are not scattered are referred to as primary transmissions and it is those transmissions that contribute the most useful information to the image. The various unscattered x-rays passing though the object are attenuated at differing levels by differing amounts and compositions of material within the object. The differences in x-ray attenuation along linear paths through the object produce an intensity pattern that comprises image information which is recorded by an image receptor.

The image receptor may be a screen having a layer of x-ray sensitive material or x-ray sensitive electronic medium. The resulting image produced by the image receptor is based on the differences in the intensity of primary x-ray transmissions detected by the receptor. To improve the image quality, the primary x-ray transmissions and any scattered x-rays that would reach the image receptor after having traveled though the body, are first passed through a grid before they are allowed to impinge onto the image receptor.

Anti-scatter grids are components of an x-ray imaging system that are placed between the imaged object and the image receptor. The purpose of the grid is to filter out any x-rays scattered by the object while permitting the unscattered (primary) x-rays to pass through. The efficiency of the grid is dependent on two factors, primary and the scatter transmission. Primary transmission should be as high as possible, while scatter transmission should be as close to zero as possible. It is understood that, quantitatively speaking, the scattered x-rays degrade the image contrast by a factor approximately equal to (1-SF) where SF is the scatter fraction of the total radiation transmitted through the body. The scatter fraction SF is defined as:

$$SF = \frac{S}{S + P}$$

where S and P are the intensities of the scattered and primary radiations incident on the image receptor, respectively. Most conventional anti-scatter grids are considered linear grids as they are fabricated as a linear array of thin lead foil ribbons. Generally, the performance of an anti-scatter grid in this respect is given by the Contrast Improvement Factor (CIF):

$$CIF = \frac{{C}_{g}}{{C}_{o}} = \frac{1 - SF}{1 - \frac{S \times {T}_{s}}{S \times {T}_{s} + P \times {T}_{p}}}$$

where C_{g} and Cₒ are the image contrasts with and without the grid, Tₛ and Tₚ are the transmissions of scatter and primary radiation by the grid, respectively. The ribbons are held in place by an x-ray transparent material that provides rigidity and that aligns the foils so that their surfaces converge to a focus line located at a specific distance above the top surface of the grid. X-ray transparent protective covers are usually provided over the top and bottom surfaces of the grid.

Primary transmission is an important factor because the lower the value the greater the radiation dose to a patient imaged with the grid. Primary transmission is determined by two factors, 1) the fraction of the x-ray incident surface occupied by the lead foils and 2) by the x-ray absorption of the supporting interspace material between the lead strips and by the protective covers. An ideal grid operated at its focus distance would have a primary transmission determined almost entirely by factor 1, with little absorption by the covers and interspace materials.

Scatter transmission depends on the thickness of the lead foils and on the geometry of the foils and the space between them. To a first order, the scatter transmission is reduced by increasing the thickness of the lead foil and by increasing the ratio of the foil height to the space between foils (grid ratio). Scatter transmission is further reduced by stacking two linear grids (crossed grids) with the foils in one grid orthogonal to that in the second grid although with conventionally designed aluminum interspace grids this reduces primary transmission thus requiring an increase in dose to the patient. Scatter transmission is also influenced by the attenuation properties of the interspace material. An explanation with diagrams is set forth in U.S Patent Publication No. 2013/0272505, published on Oct. 17, 2013.

### SUMMARY OF THE INVENTION

The present invention provides an improved anti-scatter grid and a method for the manufacture of, a linear focused anti-scatter grid with greater primary transmission and smaller scatter transmission compared to conventionally manufactured linear grids.

In accordance with the present invention the grid comprises a series of walls formed of a material that is capable of absorbing such high energy radiation, the walls being so placed and aligned as to define the channels in such a manner as to focus any radiation passing through the channels on a single remote line or point. The walls absorb any scattered radiation, which travel transversely to the focused radiation, but may enter the grid with the focused radiation. The radiation absorbent walls are preferably supported by a frame, generally rectangular in outline, and in a preferred embodiment of the present invention by an interstitial structure, extending between and connecting the energy absorbing walls, and formed of a polymer; the polymer is preferably foamed to be at least semi-rigid, having sufficient rigidity to support and maintain the alignment of the energy absorbing walls, and having extremely low energy absorbing effect. Because the primary transmission of this invention is so high, the ultra high performance provided by a pair of crossed grids can be practical, with very low dose penalty. Alternatively, the liquid, uncured polymer can be mixed with glass microbubbles, hollow glass microspheres such as those manufactured by 3M, in lieu of foaming the polymer. Although the silicon in the glass walls has higher radiation absorbency than the usual polymers, as most of the volume is air the effect of the silicon is small.

The preferred embodiment of the grid of this invention can be formed with any practical grid ratio. Higher ratio grids made by conventional methods impose a radiation dose penalty due to the greater absorption in thicker interspaces. However the present invention has little radiation dose penalty from higher ratios because the foam material absorbs only a few percent over a wide range of x-ray energies. Such low absorptivity allows for the ultra high performance of a crossed grid with very low dose penalty. The preferred embodiment uses lead foils between 50 and 100 microns which produces much better scatter absorption than the thinner foils in conventional grids. More generally, the ribbons can have a thickness in the range of from 10 to 1000 microns

The radiation absorbent channel boundary can be designed to a desired or preferable state by changing the constituent elements, i.e., different atomic numbers of its elements, or the thickness or density of the absorbent layer, to better suit the absorption of x-rays of a specific range of photon energies. For example in an application using low energy x-rays such as in mammography, the absorbent layer may be only a few tens of microns thick and might include elements with atomic numbers as low as 29. Applications requiring more energetic x-rays, such as general medical radiography, may employ a thicker absorbent layer, which is preferably formed from heavy metal elements with atomic numbers above 65, such as Lead, Bismuth, Tungsten, or Tantalum.

The ribbons of metal need not necessarily be pure metal but may be a powdered material mixed with binding agents (e.g., polymers) to bind a relatively high concentration of heavy metal in the form of a fine powder, or as a compound mainly containing elements with atomic numbers greater than 28, or as an alloy. Depending on the application for which the anti-scatter grid is being used, the relatively high concentration of heavy metals may be in the range of 40% to 98% by weight. Because the channel boundaries are formed from foil under some degree of tension, some desirable, highly radiation-absorbent heavy metals, such as lead or bismuth or alloys thereof, will require reinforcement, such as by the addition of fibers or coatings (e.g., Mylar) to provide adequate tensile strength to a ribbon of the metal. This can include the use of glass fiber reinforced lead foil, or a lead foil wrapped in a thin braided weave of high tensile strength glass, nylon, polyester or other fiber materials. Alternatively, a thin tape may be adhered to the heavy metal ribbon formed, for example of Mylar or Kapton, two commercially available polymeric materials containing oxygen or nitrogen, respectively. As a further alternative, the heavy metal ribbon can be plated or otherwise bonded to a thin metal strip, preferably high tensile strength steel or stainless steel foils. The steel foil need only be 20-50 microns thick. As already described with respect to FIG. 1, the focused grid 16 of the present invention has a plurality of focused channels that allow unscattered primary x-ray beams having passed through the object 12 to impinge upon the image receptor 14, and thus form a clear, focused image.

One preferred embodiment of the focused grid of the present invention comprises an enclosed frame, comprising at least a pair of opposed side pieces, each supporting and positioning a ribbon of the material forming the grid walls. In one embodiment, an assembly frame base member comprising two parallel assembly frame members support parallel rows of small pins 6 on opposite sides of the assembly frame base. Each corresponding pair of pins in the two frame members provide support for a loop of a heavy metal foil, or ribbon, extending across the assembly frame base. There are preferably an equal number of pins in each row and pairs of pins on opposite rows are parallel to each other.

In another embodiment, each side piece is provided with a plurality of slots, or other openings, so spaced and disposed as to hold, preferably at each end, the material forming the walls defining the channels, in the proper alignment. The slots are so disposed relative to each other as to cause the wall materials held in the slots, to be in a configuration to focus any radiation impinging on one face of the grid to converge at a focus point, or line, beyond the opposing surface of the grid.

Preferably, the grid is formed of a series of interconnected and mating modules, each module being substantially identical to the other modules. In one such embodiment, each module is essentially a ribbon, or plate, of the radiation absorbent heavy metal material, held in a frame so as to maintain their juxtaposition relative to each other and to the radiation source and the imaging device. In another such embodiment, the ribbon, or plate, of the radiation absorbent heavy metal material is secured to one side of a suitably shaped support formed of a radiation transparent material, also preferably held in a suitable frame, as above.

In each preferred embodiment there is extending between, and defined by, the radiation absorbent walls, a substantially radiation transparent material, which most preferably, is only, or primarily, air, the material most transparent to x-rays. Alternatively, as a means of providing additional structural support and rigidity to the radiation absorbent walls, extending between and attached to at least one of the immediately adjacent pair of defining walls is a solid support material that is also substantially transparent to x-radiation, such as a hydrocarbon polymer or carboxylated hydrocarbon polymer, if the polymer is thick enough to completely fill the channel between the walls, the polymer is more preferably foamed to further increase primary radiation transmission. The grid design most preferably contains primarily air within the channels, so that transmission of primary radiation (Tₚ) through the grid is maximized, thus allowing the total radiation dose to the patient to be lower, as compared to conventional aluminum- or steel-supported grids.

A foam could consist of a polymer incorporating glass microbubbles; these are hollow glass microspheres such as those manufactured by 3M. Although the silicon in the glass walls is actually not ideal, as the walls are extremely thin, so that most of the volume of the microspheres is air, the effect of the silicon is small, and the possible vagaries of a foaming chemical reaction are avoided.

The thickness of the heavy metal, x-ray absorbent walls defining the channels and the depth of the channels (and thus the length, L, of the walls) can be varied to optimize primary transmission and reduce or eliminate transmission of the scattered radiation, for a given radiation energy.

One preferred method of the present invention comprises the steps of forming a preferred grid frame by forming the frame sides, by casting or molding, of for example, aluminum or steel or a high strength polymer. In the method of forming one preferred embodiment of the frame, high precision machining of the aluminum or steel or rigid polymer frame sides, produces a series of aligned slits on opposite sides of the frame. The planes containing the center lines of the pairs of opposed slits along the opposing frame sides, are so aligned and juxtaposed, as to converge at a line on the horizontal plane of the x-ray tube focus, as depicted in FIG. 1.

The slits on opposite sides of the frame are precisely aligned so that slits on opposite sides are in the same planes orthogonal to the sides of the frame in which the slots are formed. The walls can be formed of thin ribbons of heavy metal foils held tightly in tension across the frame by the opposed slits. One embodiment is essentially a conventional linear grid where the metal foil ribbons form planes that extend from one edge of the frame to the other. In this embodiment the planes of all ribbons converge to a line through the x-ray focus.

A second related embodiment is based upon the first embodiment, except that a second similar frame is positioned over the first but with the slits and ribbons orthogonal to those of the first layer. This design results in what is effectively a crossed linear grid, which further reduces scatter radiation striking the imaging surface and results in a further improved image. The grid ratio is the ratio of channel depth to diameter, which can be 3:1 to 20:1, and is preferably between 5:1 and 16:1.

In another group of preferred embodiments, the focused grid of the present invention comprises an enclosed frame, comprising at least a pair of opposed side pieces, each supporting and positioning a ribbon of the material forming the grid walls, and each side piece provided with a plurality of slots, or other openings, so spaced and disposed as to hold, preferably at each end, the material forming the walls defining the channels, in the proper alignment. The slots are so disposed relative to each other as to cause the wall materials held in the slots, to be in a configuration to focus any radiation impinging on one face of the grid to converge at a focus point, or line, beyond the opposing surface of the grid.

Preferably, the grid is formed of a series of interconnected and mating modules, each module being substantially identical to the other modules. In one such embodiment, each module is essentially a ribbon, or plate, of the radiation absorbent heavy metal material, held in a frame so as to maintain their juxtaposition relative to each other and to the radiation source and the imaging device. In another such embodiment, the ribbon, or plate, of the radiation absorbent heavy metal material is secured to one side of a suitably shaped support formed of a radiation transparent material, also preferably held in a suitable frame, as above.

In each preferred embodiment there is extending between, and defined by, the radiation absorbent walls, a substantially radiation transparent material, which most preferably, is only, or primarily, air, the material most transparent to x-rays. Alternatively, as a means of providing additional structural support and rigidity to the radiation absorbent walls, extending between and attached to at least one of the immediately adjacent pair of defining walls is a solid support material that is also substantially transparent to x-radiation, such as a hydrocarbon polymer or carboxylated hydrocarbon polymer, if the polymer is thick enough to completely fill the channel between the walls, the polymer is more preferably foamed to further increase radiation transmission. The grid design most preferably contains primarily air within the channels, so that transmission of primary radiation (Tₚ) through the grid is maximized, thus allowing the radiation dose to the patient to be lower, as compared to conventional aluminum- or steel-supported grids.

The thickness of the heavy metal, x-ray absorbent walls defining the channels and the depth of the channels (and thus the length, L, of the walls) can be varied to optimize primary transmission and reduce or eliminate transmission of the scattered radiation, for a given radiation energy.

One preferred method of the present invention comprises the steps of forming a preferred grid frame by forming the frame sides, by casting or molding, of for example, aluminum or steel or a high strength polymer. In the method of forming one preferred embodiment of the frame, high precision machining of the aluminum or steel or rigid polymer frame sides, produces a series of aligned slits on opposite sides of the frame. The planes containing the center lines of the pairs of opposed slits along the opposing frame sides, are so aligned and juxtaposed, as to converge at a line on the horizontal plane of the x-ray tube focus, as depicted in FIG. 1.

The slits on opposite sides of the frame are precisely aligned so that slits on opposite sides are in the same planes orthogonal to the sides of the frame in which the slots are formed. The walls can be formed of thin ribbons of heavy metal foils held tightly in tension across the frame by the opposed slits. One embodiment is essentially a conventional linear grid where the metal foil ribbons form planes that extend from one edge of the frame to the other. In this embodiment the planes of all ribbons converge to a line through the x-ray focus. A second related embodiment is based upon the first embodiment, except that a second similar frame is positioned over the first but with the slits and ribbons orthogonal to those of the first layer. This design results in what is effectively a crossed linear grid, which further reduces scatter radiation striking the imaging surface and results in a further improved image. The grid ratio is the ratio of channel depth to diameter, which can be 3:1 to 20:1, and is preferably between 5:1 and 16:1.

The method of producing the grid, in accordance with this invention results in an improved product and avoids the cost of expensive tensioning apparatus being required for the final grid.

### BRIEF DESCRIPTION OF THE SEVERAL VIEWS OF THE DRAWING

FIG. 1 shows an isometric view of the finished grid of the present invention, looking down on the top cover.

FIG. 2 shows a partially exploded, isometric view of the side frame, and the interior of the finished grid of FIG. 1, adjacent the frame member;

FIG. 3 shows an isometric view of one frame member of the assembly frame unit, showing the array of pins for holding the heavy metal strips in place;

FIG. 3A shows a partial, magnified isometric view of one edge of the frame member of FIG. 3, showing a portion of the array of pins for holding the lead strips in place;

FIG. 4 shows an isometric view of a partially constructed assembly frame unit, including the opposing frame members holding the two parallel series of pins for supporting the heavy metal strips used during manufacture of the grid;

FIG. 5 shows an isometric view of additional members of the assembly frame unit, including the movable series of pins and the assembly frame member supporting the pins, plus the thin, rigid bottom cover of the finished grid;

FIG. 6 shows a magnified partial isometric view looking down on the movable end of the of the assembly frame, including the movable series of pins and including the thin, rigid bottom cover, with one of the heavy metal ribbon loops placed around one of the pins;

FIG. 7 shows another highly magnified, partially broken away, isometric view of the movable end of the assembly frame, from a slightly different angle than in FIG. 6, with the heavy metal ribbon loops placed around each of the pins shown;

FIG. 8 shows an isometric view of the completed assembly frame with the rigid assembly frame cover secured thereover;

FIG. 9 shows a magnified partial isometric view of the movable end of the fully-assembled frame;

FIG. 10 shows an isometric view of the completed assembly frame with the rigid assembly frame cover secured thereover, and after suitable tensioning, foamable, polymerizable mixture is introduced through an injection port under pressure;

FIG. 11 shows another magnified, partial isometric view of the movable end of the assembly frame, after the rigid foam has set in place within the grid, removable covers over the pin arrays are removed to expose the fully assembled rigid grid and enabling the rigid grid to be cut from the assembly frame along the edges of the grid covers so that it can be removed as a single rigid unit;

FIG. 12 shows an isometric view of an unfolded length of a heavy metal ribbon;

FIG. 13 shows a magnified isometric view of the end of the looped ribbon of FIG. 12, showing the line where the two ends of the ribbon are joined;

FIG. 14 shows an isometric view of an assembly frame member for the array of pins for holding the lead strips in place, showing the location of the holes into which the pins fit;

FIG. 15 shows an isometric cut-away view of the support unit for the array of pins for holding the lead strips in place, of FIG. 14, along lines A_A, showing the full extent of the holes into which the pins fit, and showing how the holes are angled to support the pins in the angled manner;

FIG. 16 shows a generalized side view representation of the focused grid of the present invention placed between an object to be imaged and an image receptor:

FIG. 17 shows a side view representation of a preferred example of the focused grid of the present invention and its juxtaposition with respect to an x-ray source;

FIG. 17A shows an exploded view of a portion of the frame of the focused grid of the present invention in FIG. 17;

FIG. 18A shows a side view of a ribbon of a heavy metal foil, which forms a wall in the grid of FIGS. 17 and 18;

FIG. 18B shows a side view of the ribbon of a heavy metal foil of FIG. 19, where the ends of the foil have been folded into loops;

FIG. 18C shows a top view (from the direction of the x-ray source) of the ribbon of a heavy metal foil of FIG. 18B;

FIG. 19 shows a top view (from the direction of the x-ray source) of a portion of the frame represented in FIG. 17 holding a plurality of ribbons of a heavy metal foil of FIG. 19, the ribbons being held within the slits shown in FIG. 18A;

FIG. 20 shows a side view of the frame represented in FIG. 19 holding a ribbon of a heavy metal foil of FIG. 18B, the ribbon being held within the slits shown in FIG. 19;

FIG. 21 shows a top view (from the direction of the x-ray source) of a portion of an adjustable frame represented in FIG. 20 for holding a plurality of ribbons of a heavy metal foil of FIG. 18B, the ribbons being held within the slits shown in FIG. 17A;

FIG. 22 shows a top view (from the direction of the x-ray source) of the complete adjustable frame represented in FIG. 21;

FIG. 23 shows a top view (from the direction of the x-ray source) of a pair of the complete adjustable frames represented in FIG. 22, juxtaposed orthogonally to each other to form a grid of squares;

FIG. 24 shows a side representation of a system for forming a further improved embodiment of the ribbon forming the side walls of the grid, where the heavy metal ribbon is encased in a reinforcing tape;

FIG. 24A shows side view of the tape-encased ribbon of FIG. 24;

FIG. 24B shows a top view (from the direction of the x-ray source) of the ribbon of a heavy metal foil of FIG. 25, where the ends are held in the frame slits by crimped metal loops holding the ends of the ribbon; and

FIG. 25 shows one spring tension system of the frame of FIGS. 22 and 23, for adjusting the tension on the ribbons.

### DETAILED DESCRIPTION OF THE INVENTION

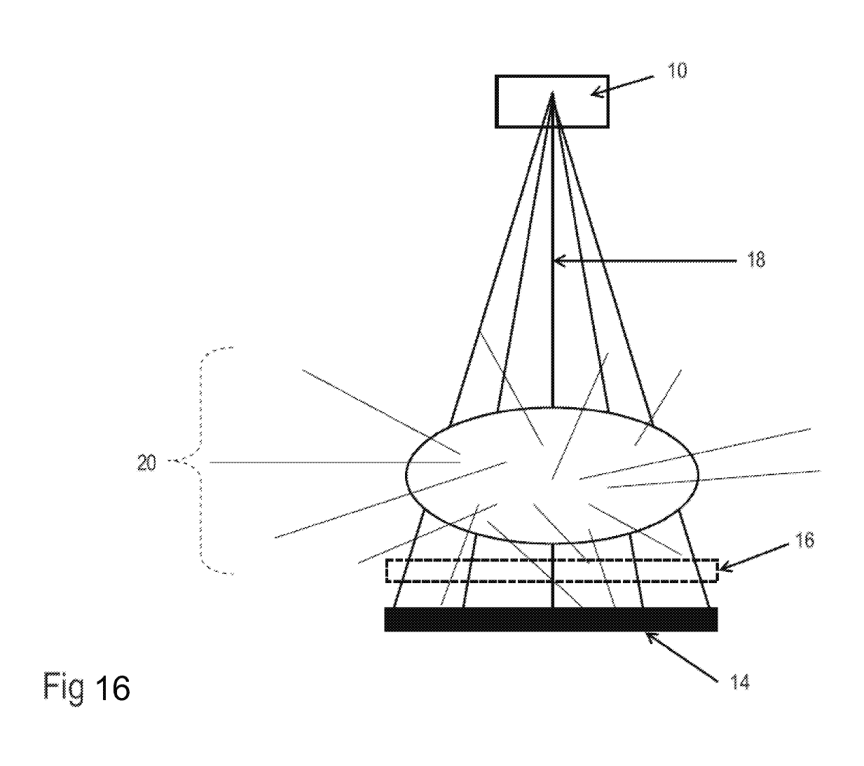

The focused grid of the present invention as described above can be used as an anti-scatter grid for x-ray imaging useful, for example, in the fields of medical and/or industrial radiography. Referring to FIG. 16, the x-rays are directed from the source (10) to pass through the object 12, to the image receptor 14, below. In passing through the object or patient, some of the x-rays are scattered 20, thus reducing the contrast in the recorded image. The grid of the present invention (1) is placed between the object or patient (12) and the image receptor (14). The grid is designed to absorb as much of the scatter radiation as possible while allowing the passage of the primary imaging x-rays. The drawings used in this application are not necessarily drawn to scale, but they are presented to clearly depict the various features and aspects of the present invention.

The x-rays used for radiographic purposes usually include electromagnetic radiation having photon energies in the range of 10 keV to 1 MeV. For ease of explanation, the beam of radiation will henceforth be described as an x-ray beam in the range described. However, it should be understood that all of the embodiments of the claimed focused grid of the present invention may be operated and function as described using electromagnetic radiation having photon energies that fall outside of the range described above, with the boundaries or walls of the channels to be constructed from material that can absorb such scattered electromagnetic radiation as may be generated.

Continuing with the description of FIG. 16, a uniform beam 18 of x-rays is directed toward a surface of the object 12 and travels through the object 12 emerging from an opposite surface of the object 12. Differences in x-ray attenuation along linear paths through the object 12 produce an x-ray intensity pattern that comprises image information recorded by the image receptor 14. The image receptor 14 can be a device such as an intensifying screen coupled with a photographic film or any layer of x-ray sensitive material or x-ray sensitive electronic medium, which through one or more steps converts the x-ray intensity pattern into a visible image or visible format.

When x-rays 18 pass through the object 12, they are attenuated by a combination of scattering and absorption. X-rays which have passed through the object 12 and are “focused x-rays” (meaning they also pass through the grid 16, following a focused path as described herein) and are also referred to as ‘primary x-rays’; the primary x-rays contribute to the formation of the image. That is, unscattered focused beams—having passed directly though the object 12—will mostly pass through the channels of the focused grid 16 of the present invention. Radiation, including x-ray radiation, which do not follow a focused path leaving the object being imaged are referred to as scattered, and scattered radiation 20 will intersect one of the metallic layers (or radiation absorbing layers) that define the channel boundaries, which are intended to absorb the scatter radiation to an extent depending on the composition and thickness of the boundary and the energy of the radiation. It must be noted that some focused x-rays, which pass in the plane of the foils will tend to be absorbed by those foils creating a shadow image of the foils in the resulting image. It is known to provide grid systems with a mechanism to move the grid during the x-ray exposure so that the image of the foils is reduced, if not eliminated, by the blurring resulting from the motion, without significantly reducing the resolution of the primary image.

The radiation absorbent channel boundary can be designed to a desired or preferable state by changing the constituent elements, i.e., different atomic numbers of its elements, or the thickness or density of the absorbent layer, to better suit the absorption of x-rays of a specific range of photon energies. For example in an application using low energy x-rays such as in mammography, the absorbent layer may be only a few tens of microns thick and might include elements with atomic numbers as low as 29. Applications requiring more energetic x-rays, such as general medical radiography, may employ a thicker absorbent layer, which is preferably formed from heavy metal elements with atomic numbers above 65, such as Lead, Bismuth, Tungsten, or Tantalum.

An x-ray transparent material (such as air, or a hydrocarbon polymer or other low molecular weight polymer which may also contain nitrogen or oxygen atoms, or even chlorine atoms) is a material through which an x-ray beam travels where the measurable intensity of the beam immediately prior to passing into the material is substantially equal to the measurable intensity of the beam immediately after exiting the material. Conversely, an x-ray absorbent material greatly reduces the amount of x-rays exiting such material compared to the strength of the x-rays that entered such material. X-rays passing through the object being imaged, and that are scattered, i.e., that do not follow a focused path through the channels of the focused grid, intersect and impinge upon the x-ray absorbent wall boundaries of the focused channels, and are thus absorbed by these walls.

The ribbons of metal need not necessarily be pure metal but may be a powdered material mixed with binding agents (e.g., polymers) to bind a relatively high concentration of heavy metal in the form of a fine powder, or as a compound mainly containing elements with atomic numbers greater than 28, or as an alloy. Depending on the application for which the anti-scatter grid is being used, the relatively high concentration of heavy metals may be in the range of 40% to 98% by weight. Because the channel boundaries are formed from foil under some degree of tension, some desirable, highly radiation-absorbent heavy metals, such as lead or bismuth or alloys thereof, will require reinforcement, such as by the addition of fibers or coatings (e.g., Mylar) to provide adequate tensile strength to a ribbon of the metal. This can include the use of glass fiber reinforced lead foil, or a lead foil wrapped in a thin braided weave of high tensile strength glass, nylon, polyester or other fiber materials. Alternatively, a thin tape may be adhered to the heavy metal ribbon formed, for example of Mylar or Kapton, two commercially available polymeric materials containing oxygen or nitrogen, respectively. The focused grid 16 of the present invention has a plurality of focused channels that allow unscattered primary x-ray beams having passed through the object 12 to impinge upon the image receptor 14, and thus form a clear, focused image.

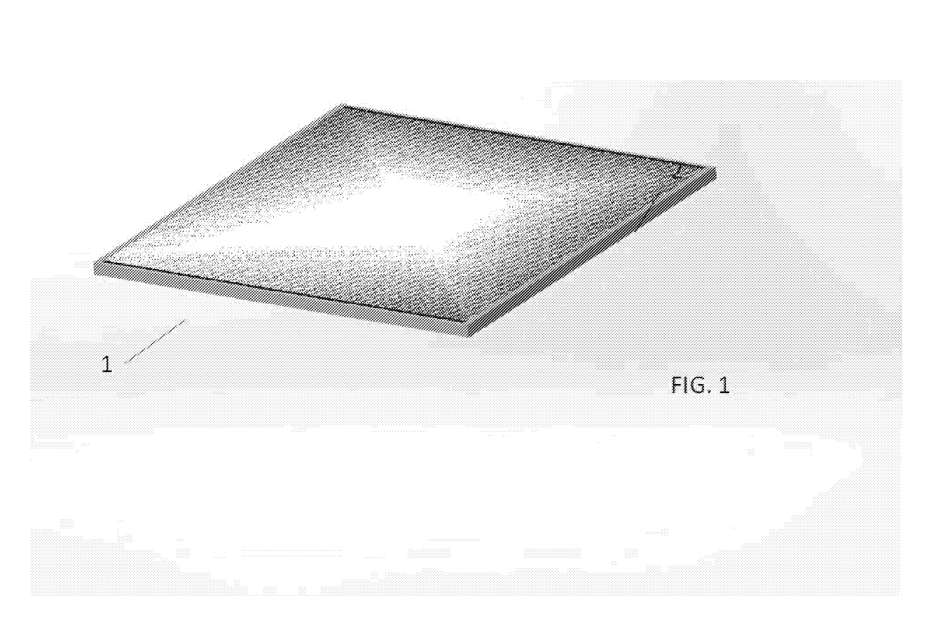

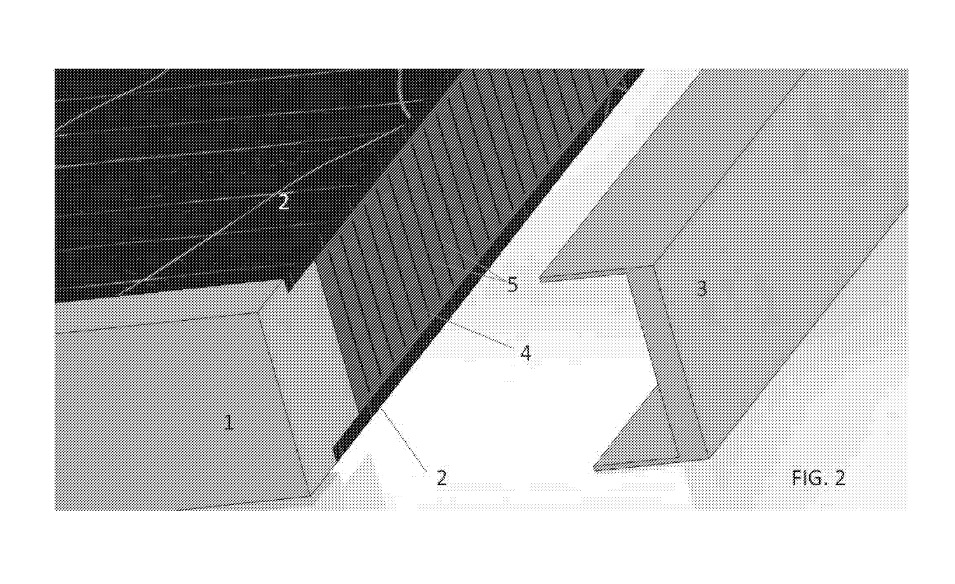

A preferred embodiment of the finished anti-scatter grid is depicted in FIG. 1. The grid consists of an aluminum frame 1 with x-ray transparent carbon fiber covers 2 on the top and bottom surfaces. The internal structure is evident in FIG. 2 with end cap 3 removed. The interior is filled with rigid, closed cell low density plastic foam such as polyurethane providing support and alignment to the lead foil suspended within the foam seen on end in cross-section. Other polymer foams include polyimide foams and the recently developed PVC foams, as well as polystyrene foams. Generally, the chemical nature of the foam is not significant as long as it does not react with lead and is formed of all low atomic weight elements, such as elements having an atomic number preferably below 18, and most preferably below 10. The carbon fiber covers have high primary transmission although similar covers are also used in conventional grid manufacture. However the polymer foam material is far superior in primary transmission than conventional aluminum or fiber based interspace material. The heavy metal strips are embedded in a rigid polymer foam 4, capable of maintaining the precise alignment and separation of the ribbon surfaces 5. The edges of the rigid foam 4 can be protected by, e.g., aluminum or solid polymer boards 3, and the top and bottom major surfaces, can be protected by, highly radiolucent material, e.g., carbon fiber plates 2, that will not substantially reduce the primary radiation passing through the grid.

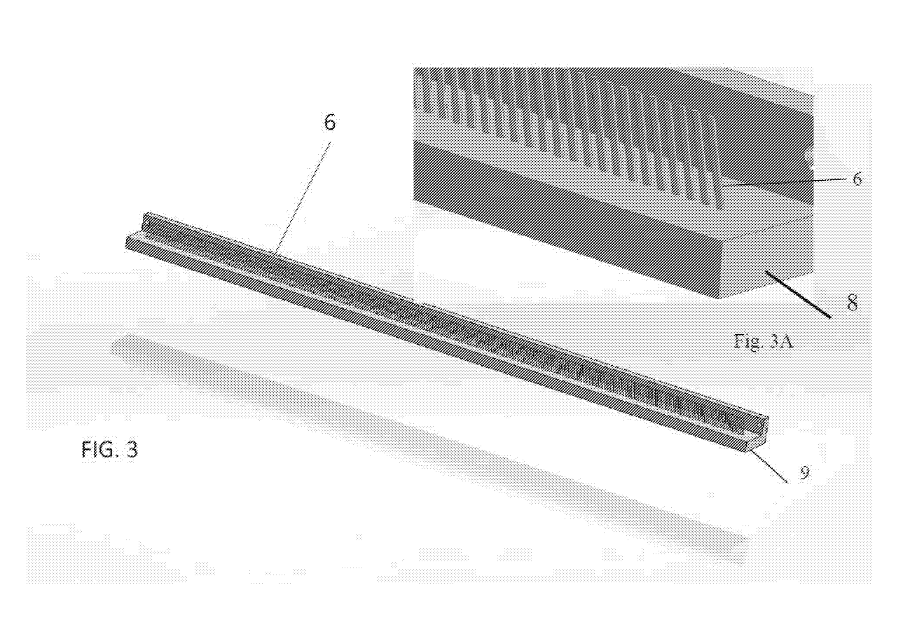

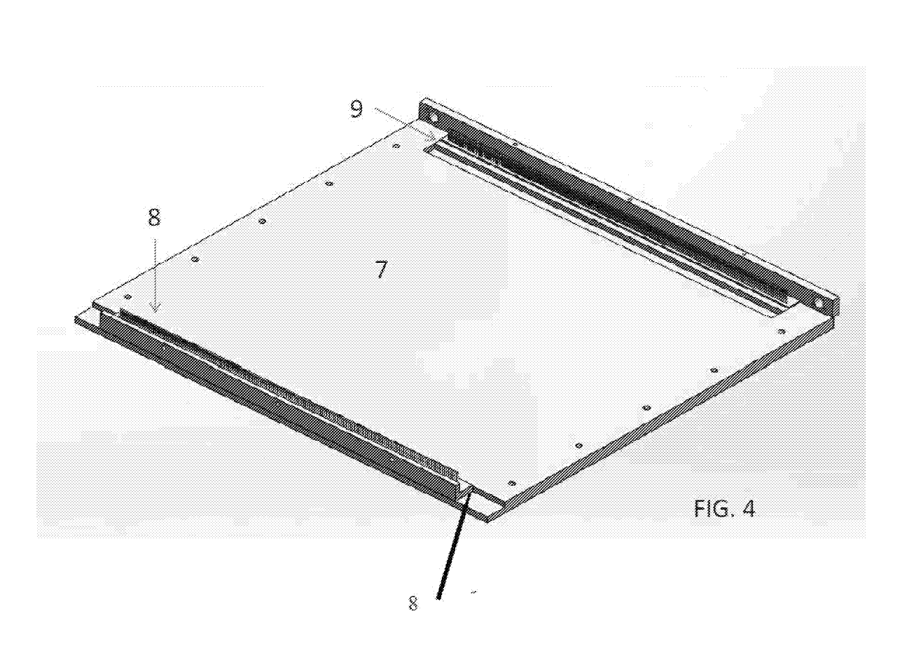

As shown in the drawings, the fabrication of the preferred embodiment begins with an assembly frame base member 7 supporting two parallel assembly frame members 8, 9 for supporting parallel rows of small pins 6 on opposite sides of the assembly frame base 7 (FIGS. 3, 3A, 4). The pins are oriented so that all axes converge to a point on the grid focus line. One row of pins 26 held by support frame 8 is fixed in position on the assembly frame 7, while the second row of pins 26 on support frame 9 can move longitudinally relative to the first row of pins and is adjustable to increase the distance from the fixed row of pins in support frame 8. There are an equal number of pins in each row and pairs of pins on opposite rows are parallel to each other. Each corresponding pair of pins 26 in the two frame members 8, 9 provide support for a loop of a heavy metal foil 13, or ribbon, extending across the assembly frame base 7. The heavy metal foil loop 13 can be reinforced by lamination on a polyester substrate to provide tensile strength. The loop is formed by cutting the cutting the metal foil ribbon, preferably reinforced with a polyester tape, to a precise length then splicing the ends together with, e.g., polyester adhesive tape 113. During assembly, loops are placed loosely over corresponding pins 6 on assembly frame members 8, 9 until all pin pairs are populated with a loop of a heavy metal ribbon.

The pins 6 can be round in cross-section, or ovoidal, or polygonal. The critical dimension is the width of the pin 6, i.e., the dimension in the direction perpendicular to the top edge of the ribbon. This dimension determines the separation of the heavy metal wall surfaces, of the grid.

As it is preferred that the tops of the ribbons be parallel and in the same plane, the pins preferably increase in length as they are located farther from the central pin, in order to compensate for the increasing incline of the pins away from the center.

Although the difference in height above the frame edge will be relatively small as a result of the incline, the best results are achieved when the top edges of the ribbons are in the same plane, when tensioned by moving apart the two frames supporting the two sets of pins.

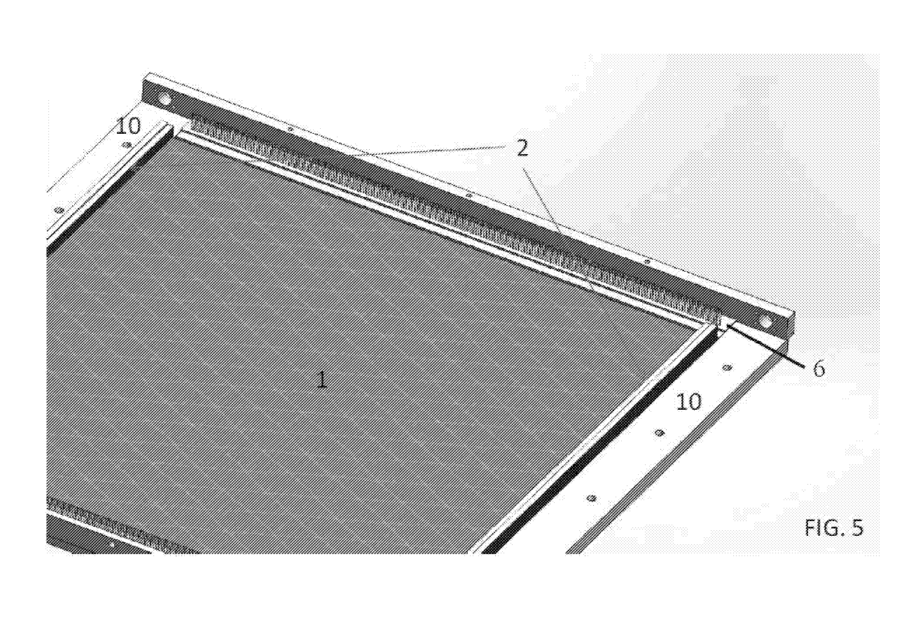

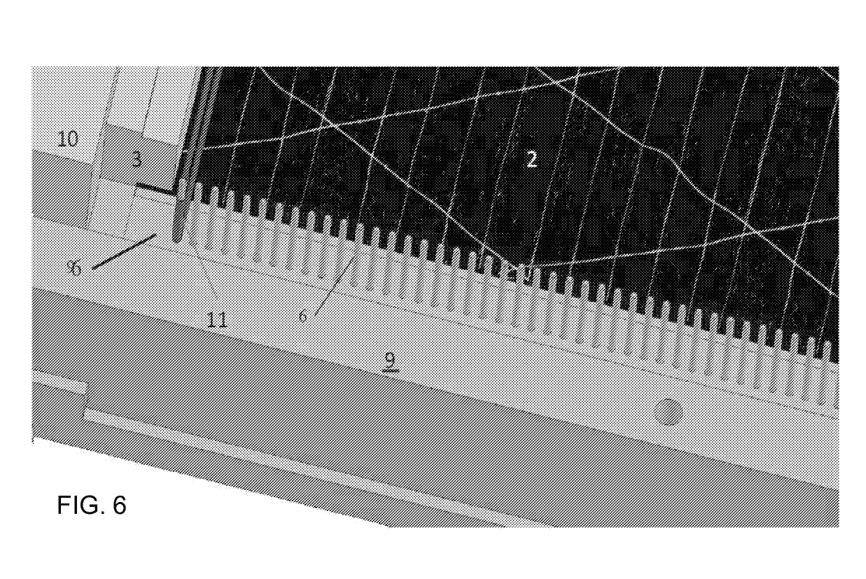

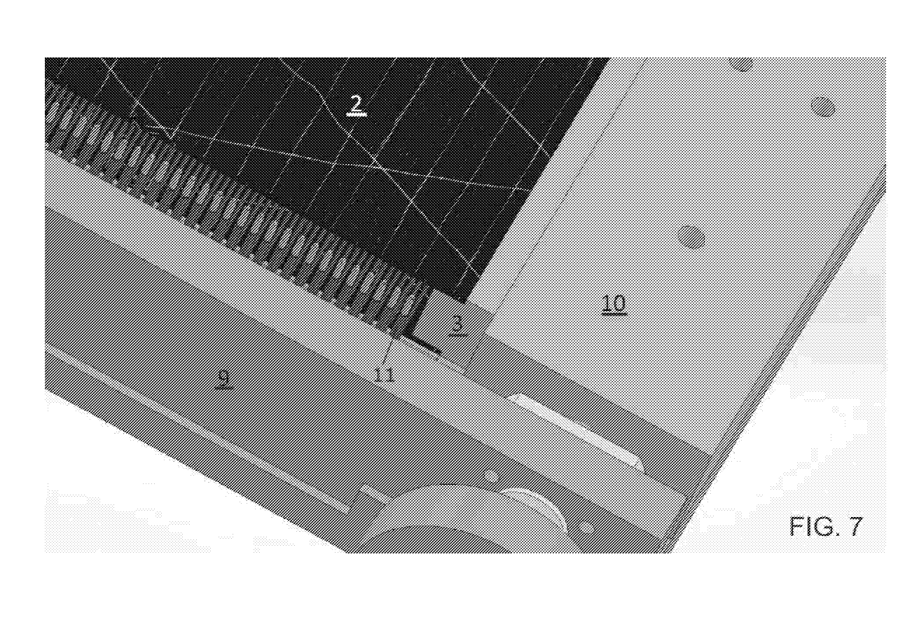

Prior to placement of the ribbon loops 11 on each pin 6, supporting blocks 10 are added to the assembly frame FIG. 5. The carbon fiber grid bottom cover 2 is added then the side frame supports 3 with adhesive holding the cover 2 to the side supports 3. The ribbon loops 13 are then placed over corresponding pins 6, FIG. 6. After all ribbon loops are installed, the top carbon fiber cover 2 is installed with adhesive to the side frame supports 3, FIG. 7.

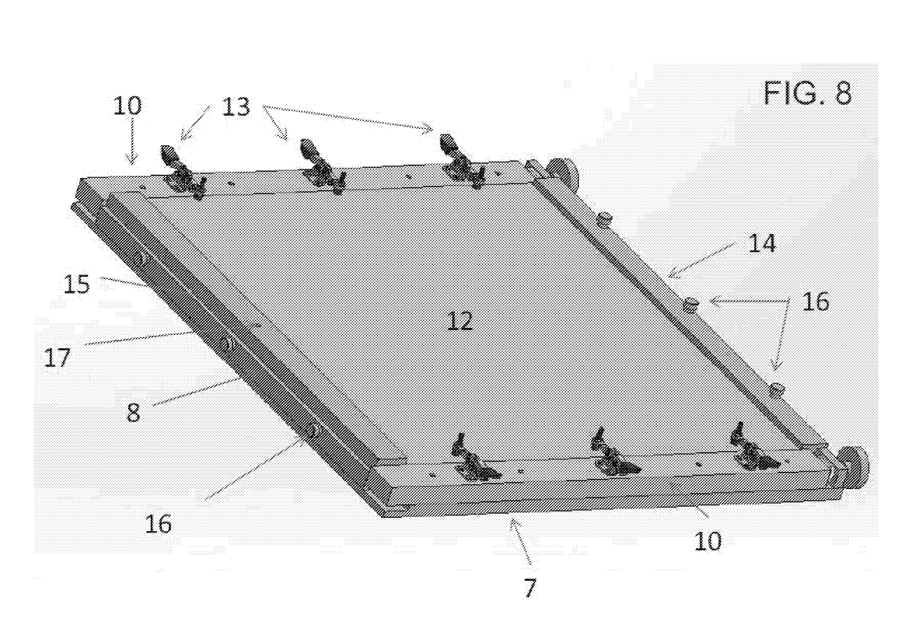

Referring to FIG. 8 the rigid assembly frame cover 12 is placed over the grid top cover and clamped in place with clamps 13. A cover 14 is added over the adjustable pin array and a cover 17 over the fixed pin array 15 and fixed in place with thumb screws 16 into threaded openings 116 in assembly frame 8.

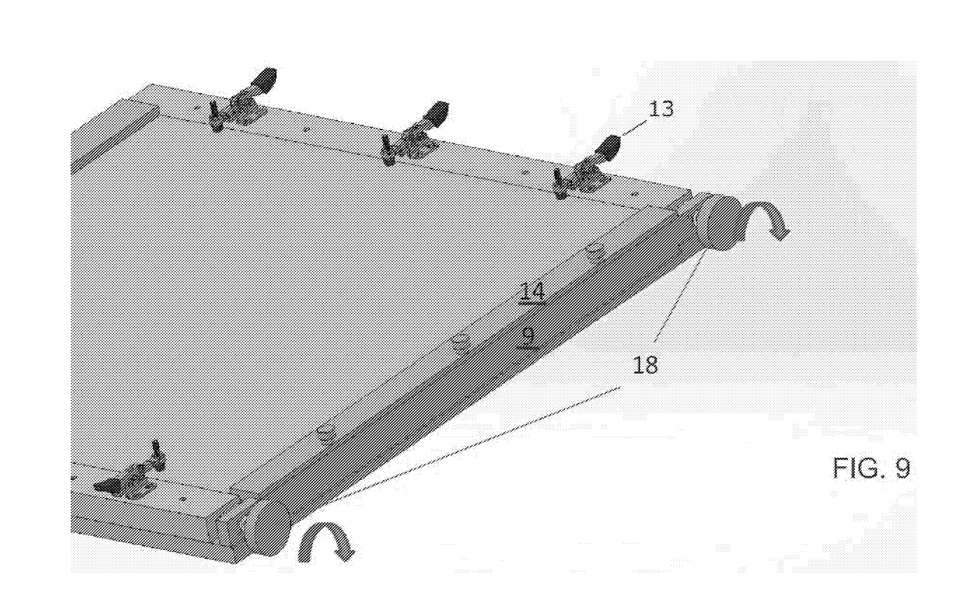

Referring to FIG. 9, a pair of knobs 18 attached to screws are employed to pull outward on adjustable pin array 9 together with its cover 14 to tension the foil loops 11 and ensure that they are in proper alignment. Slots (not visible) within in covers 14 and 15, aligned with the pins on frames 9,8, respectively, support the upper ends of the tensioning pins 6 in both arrays so that they do not bend when the tensioning force is applied.

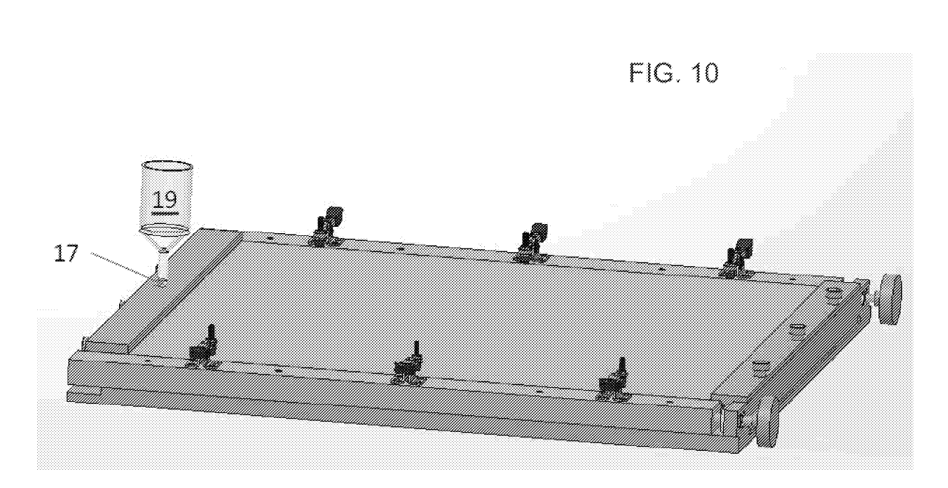

After suitable tensioning, the support frame 9 is locked in position to maintain the desired tension, and the two part polyurethane mixture is introduced through the injection port 17 using a mixing nozzle 18 under pressure, see FIG. 10.

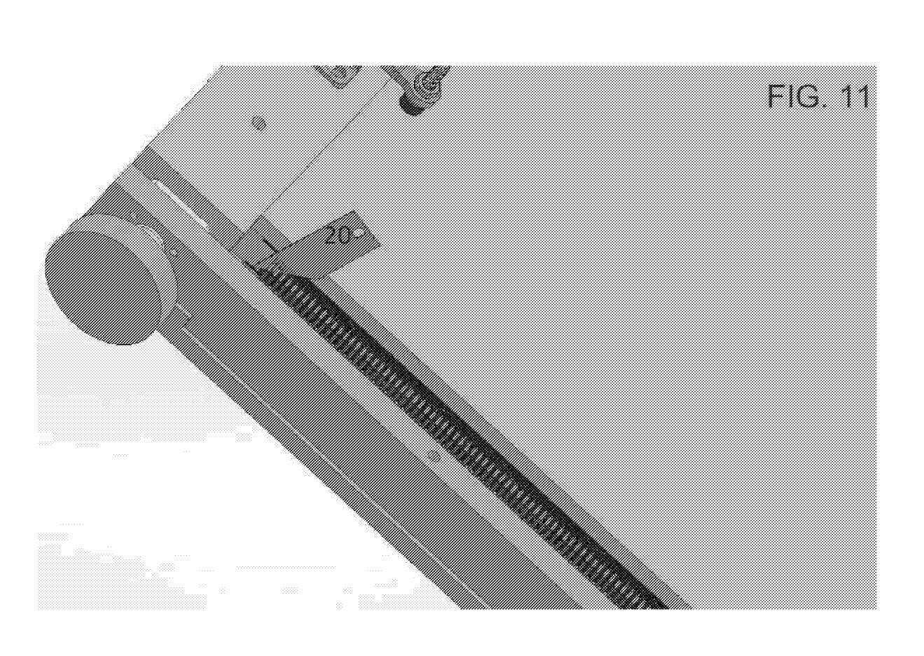

Referring to FIG. 11, after the foam has cured in place within the grid, the covers on the pin arrays are removed and the grid is cut from the assembly frame along the edges of the grid covers using a knife 20 or other cutting instrument.

The grid is removed from the assembly form appearing as in FIG. 2 and end plates 3 and optionally side plates, are added, attached with adhesive. The final finished grid is as shown in FIG. 1.

Prior to initial assembly the surfaces of the pin arrays, pins covers and other components of the assembly frame are coated with a suitable release agent to prevent adhesion of urethane foam and to facilitate cleaning for assembly of the next grid in production.

In the preferred embodiment depicted in FIG. 1, the polymer foam material is far superior in permitting primary radiation transmission compared to conventional aluminum supports or fiber based interspatial material. Depending upon the extent of the foaming, it is almost completely air. An important factor in this design is that the foamed polymer, e.g., polyurethane foam, within the spaces between the lead/polyester foil ribbons does not generate sufficient pressure force, as it polymerizes and foams, so as to cause distortion of the foils, and thus disturbing the focusing. Specialty foams designed for construction insulation of spaces around windows and doors are designed to produce minimal expansion and minimal force and should be suitable for this application. Dow Chemical Great Stuff Pro Window and Door Insulating Foam has the appropriate expansion properties while foams designed for filling of large cracks and spaces in construction that generate large expansion forces would not be suitable. An example of such a suitable foam is described in U.S. Pat. No. 6,410,609. Although urethane foam, for example, can be designed to achieve a variety of densities when fully expanded, densities less than 0.02 g/cm³ are considered to be optimal, by providing adequate mechanical support to the lead foil ribbons while producing minimal absorption of primary transmission x-rays.

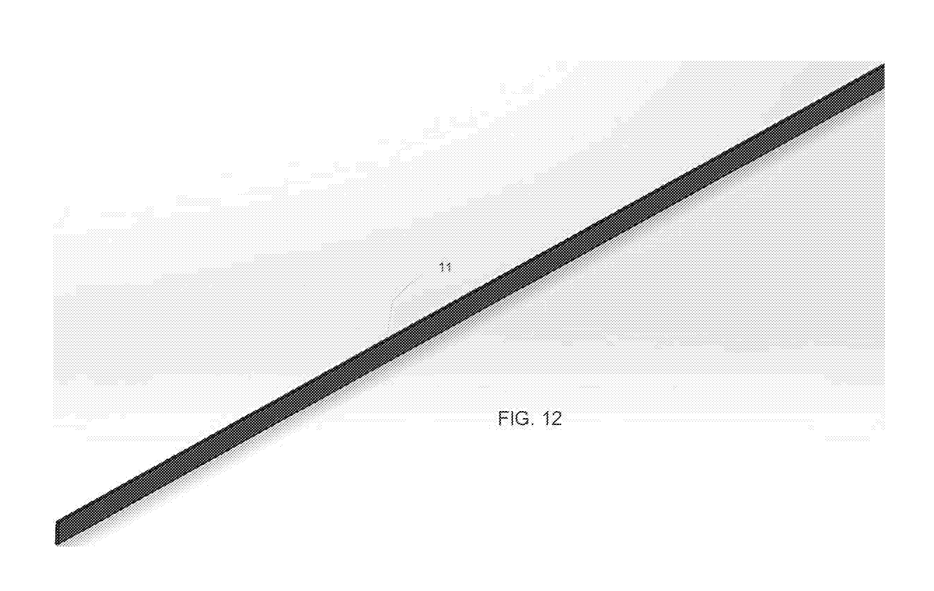

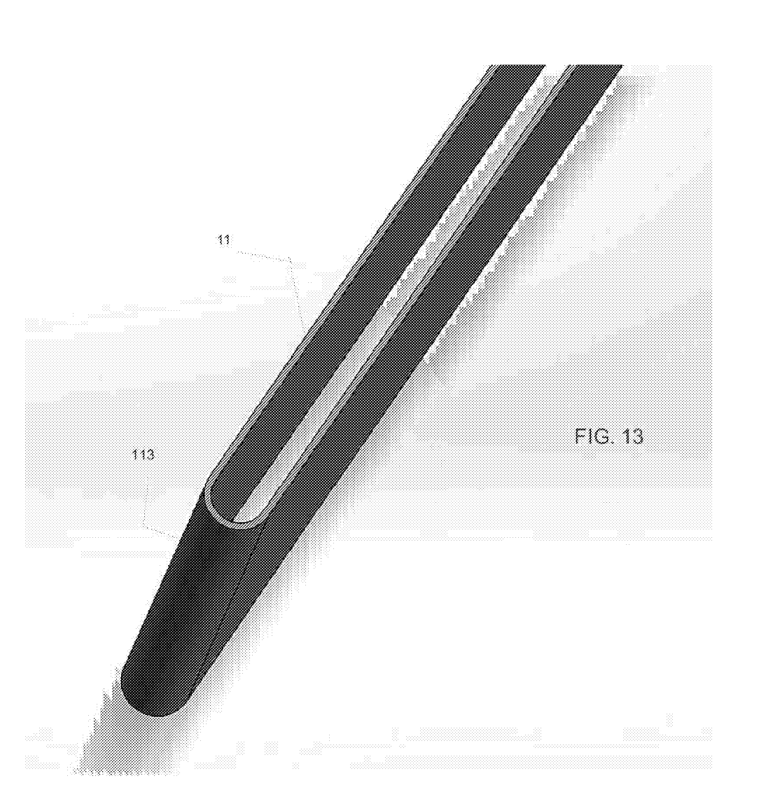

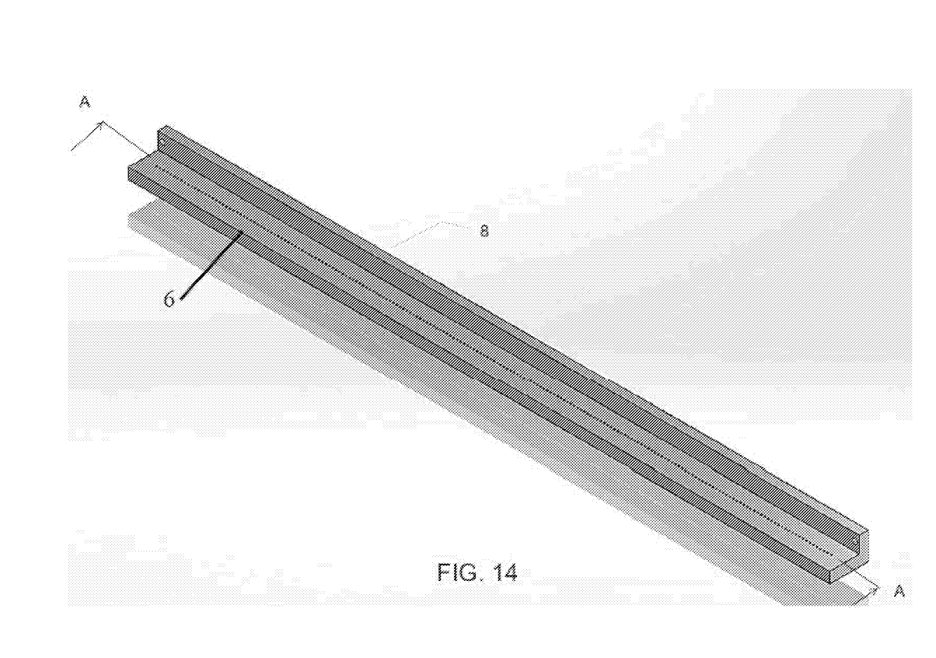

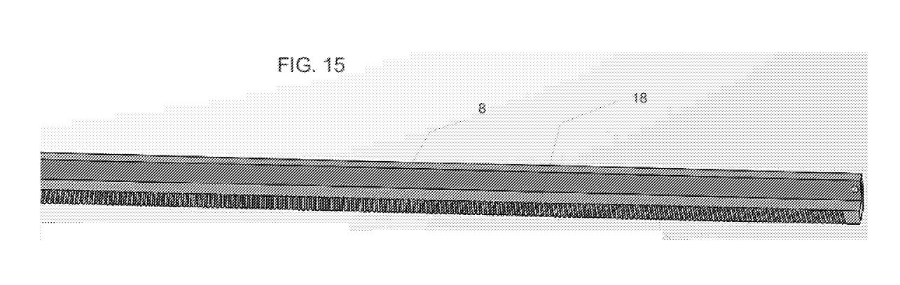

An advantage of the rigid grid of the present invention is that it maintains the linearity and juxtaposition of the grid ribbon surfaces without requiring additional hardware. In preparing the metal strips for tensioning and immersion into the rigid polymeric foam, lengths of the heavy metal ribbon are cut by a precision cutter to the exact same lengths. Each such precisely cut length is formed into a loop and the ends secured together to form the loop as shown in FIGS. 12 and 13.

A pair of series of pins 6, accurately aligned and in angularly juxtaposed relationships are held in a pair of opposing, parallel frame members 8, 9. Each frame member 8, 9 is provided with a series of holes 26, which are precisely formed and spaced to firmly hold the pins 6 in the desired juxtaposed relationships. The pins 6 are all precisely cut to the desired lengths and the diameter of each pin, and thus of the internal diameter of the hole, is equivalent to the desired foil spacing. The distances between the holes 26 are precisely the same and equivalent to the desired foil spacing plus twice the thickness of the ribbon walls 11. The holes 26 are formed to identical depths but angled such that the central axis of each of the holes, and thus of each of the pins 6 held therein, converge to the focus line, i.e., a line parallel to the surface of the grid midline that is located the focus distance above the grid surface. For example the central pins on each of frames 8, 9 are preferably perpendicular to the frame members. Typical general radiography grids, used in clinical radiography, are focused to 1000 or 1800 mm, representing the distance between the grid and source of the primary radiation.

Suitable polymeric foams include not only polyurethane foams, but also foams, for example, from polyimides, among other polymer materials.

In another group of preferred embodiments, the focused grid of the present invention comprises an enclosed frame, comprising at least a pair of opposed side pieces, each supporting and positioning a ribbon of the material forming the grid walls, and each side piece provided with a plurality of slots, or other openings, so spaced and disposed as to hold, preferably at each end, the material forming the walls defining the channels, in the proper alignment. The slots are so disposed relative to each other as to cause the wall materials held in the slots, to be in a configuration to focus any radiation impinging on one face of the grid to converge at a focus point, or line, beyond the opposing surface of the grid.

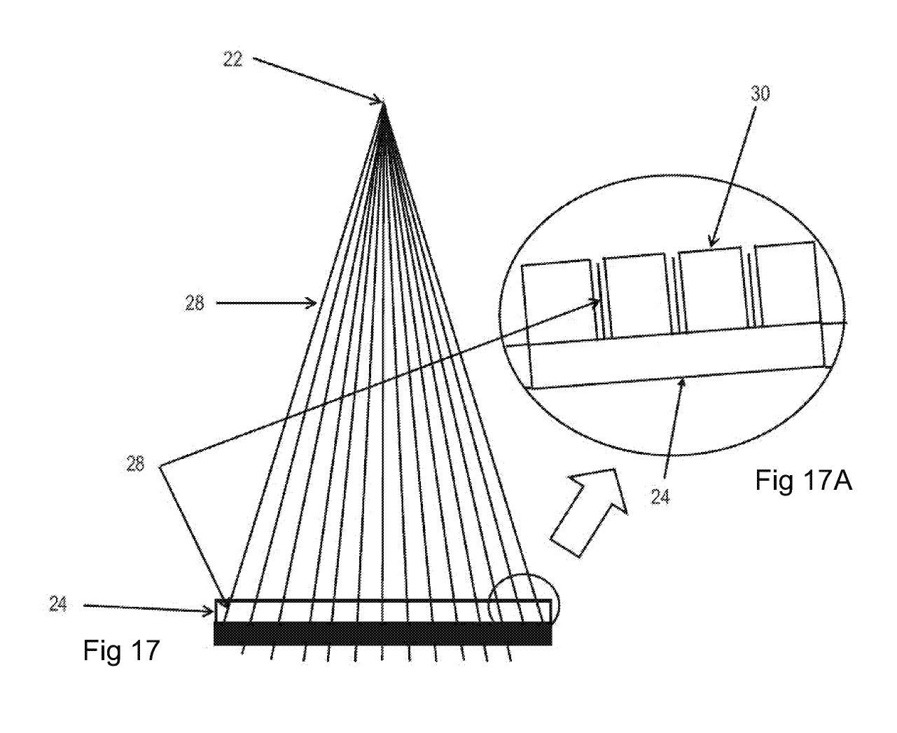

Referring now to the embodiments of FIGS. 17-25, the basic concept of the frame with slits to support, hold and align the metal foils, is shown. In FIGS. 17-19, the slits 28 and 44, respectively, are formed along the margins of two opposing sides 24 of the open frame, are in planes that are aligned along straight line paths 26 to the x-ray source 22, and extend fully through the wall of each end of the frame 24. A thin heavy metal foil ribbon 30 is stretched between a pair of the narrow slits 28 on opposite sides of the frame 24, so as to lie within the aligned plane extending between the slits.

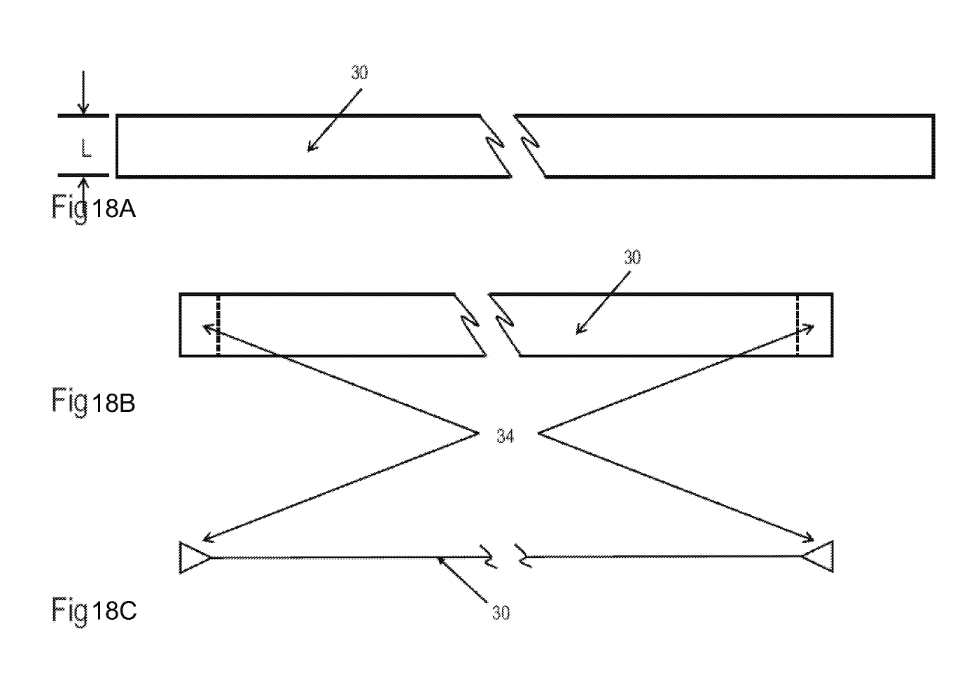

Referring now to FIGS. 18A-C, the details of the foil ribbons are shown. The foils are made of suitable materials with adequate tensile strength and high atomic number such as tungsten, tantalum or alloys thereof, fabricated into foils of the desired thickness, and cut to ribbons of height ‘d’ and sufficient length 32 to traverse the distance between the opposite sides of the open frame, including the opposing slits FIG. 18A. Sufficient ribbon length is cut to permit the folding of ends into a loop with a triangular cross-section 34, shown in side view in FIG. 18B and in edge view in FIG. 18C. The purpose of the folds 34 at the ends of foil ribbons 30 is to prevent the ribbon from being pulled through the bracket slits 28 when under tension. The shape of the folds at the ends of the ribbons 30 is intended to maintain the foil precisely aligned with the centerline of the bracket slit and thus with the x-ray tube when the ribbon is held under tension.

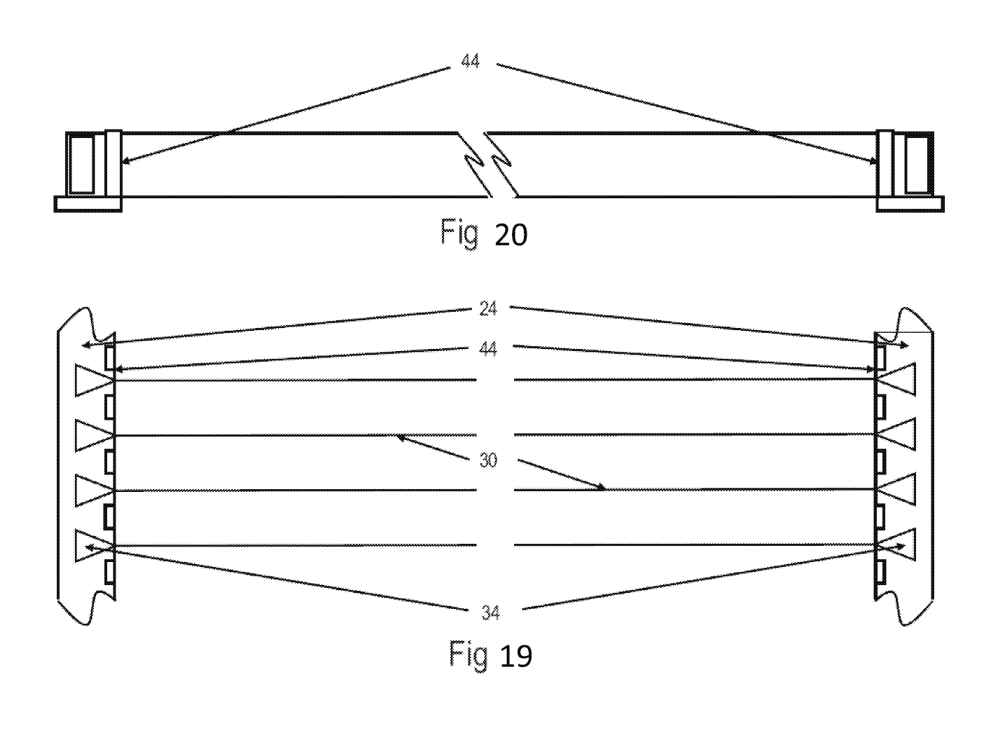

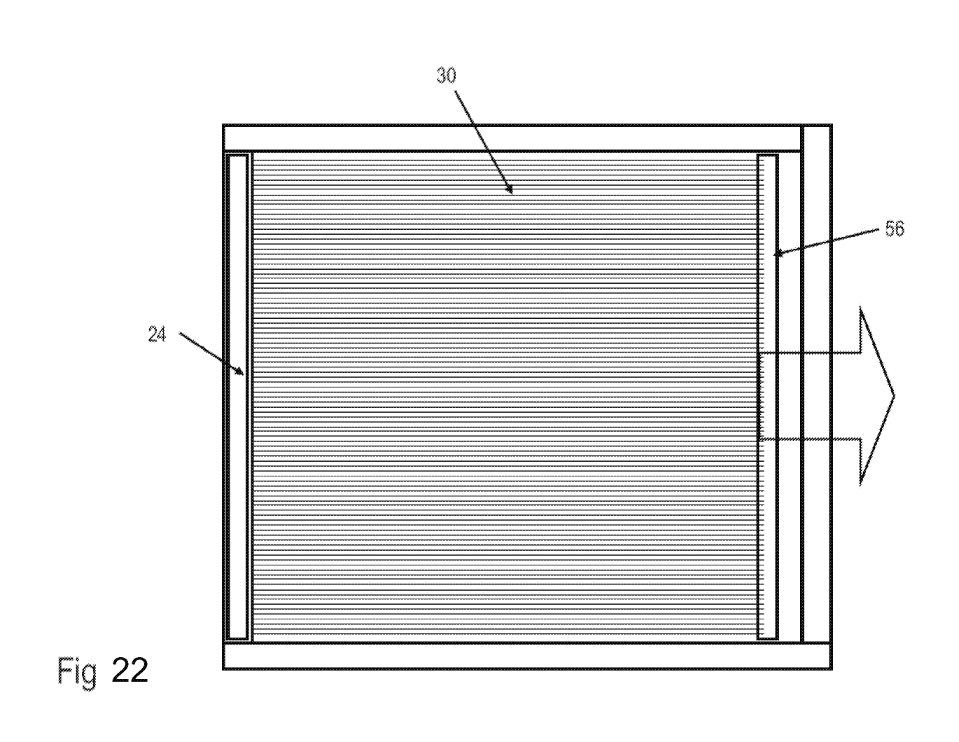

Referring now to FIGS. 19 and 21, the foil ribbon 30 with folded ends 34 is inserted into corresponding opposed slits in brackets 44 mounted on opposite sides of the frame 24. As can be seen, the folded loops at the ends are too large to fit through the slits 28, thus preventing the ribbon from slipping through the bracket when the frame 24 is sized to produce tension on the ribbons. Referring to the preferred embodiment of FIG. 21, the foil ribbons are held between a fixed bracket 44 to the outer frame 24 while a second movable set of brackets 56 can be adjusted outward to pull the ribbons into tension across the frame, so as to insure they are aligned in the desired focusing plane.

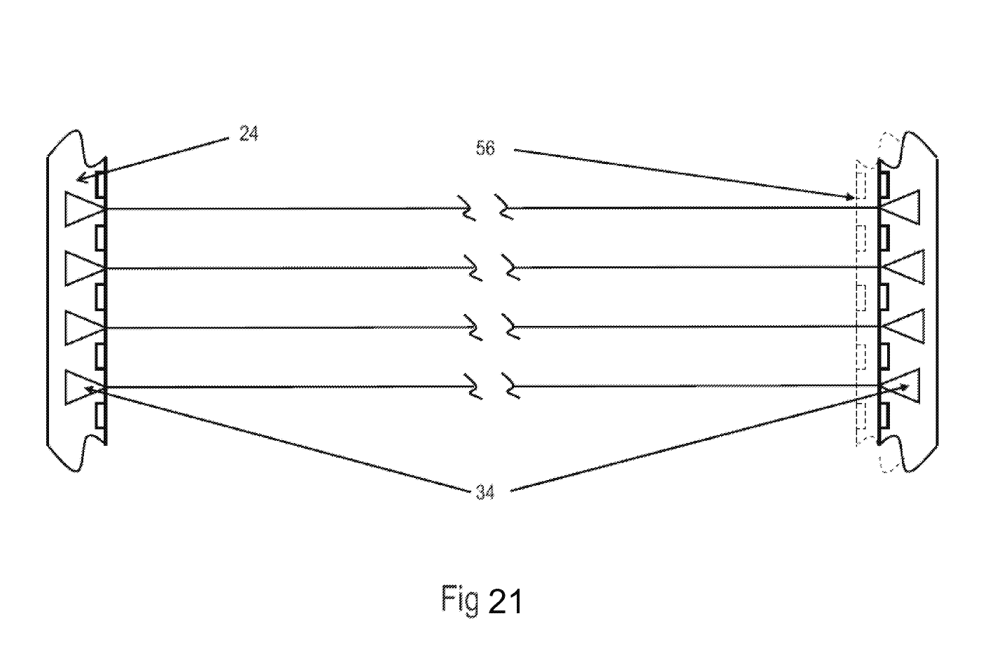

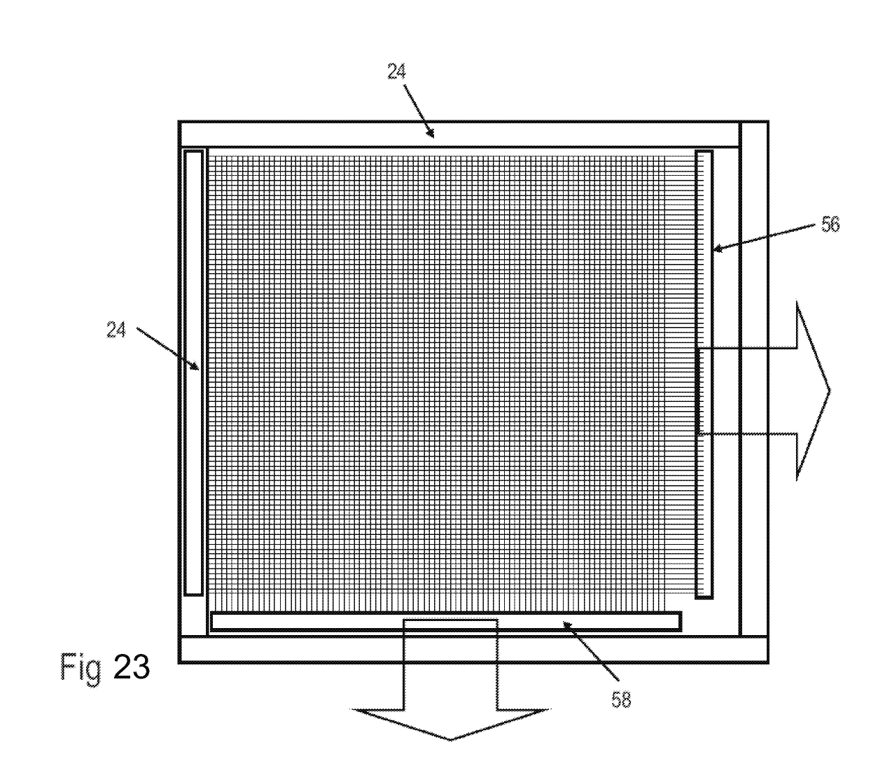

In one embodiment the slits are present only on two sides of the grid frame (FIG. 22), creating narrow focused channels as in conventional linear grids. In this embodiment, the foil ribbons 30 are tensioned between a fixed bracket 62 and an adjustable, movable bracket 56 on the opposite side of the frame. Referring back to FIG. 20 an important aspect of this design is that the shape of the folds 34 at the ends of the ribbons 30 ensures that when under tension each foil 30 is precisely aligned with the centerline of the bracket slit and thus with the x-ray source focus.

In this embodiment the scatter rejection capability would be similar to a conventionally fabricated linear grid with the same grid ratio, the same metal thickness and composition of channel walls, except that in accordance with this invention, the presence primarily of air in the channels between the metal ribbons ensures that the transmission of the primary radiation is substantially unimpeded and therefore superior.

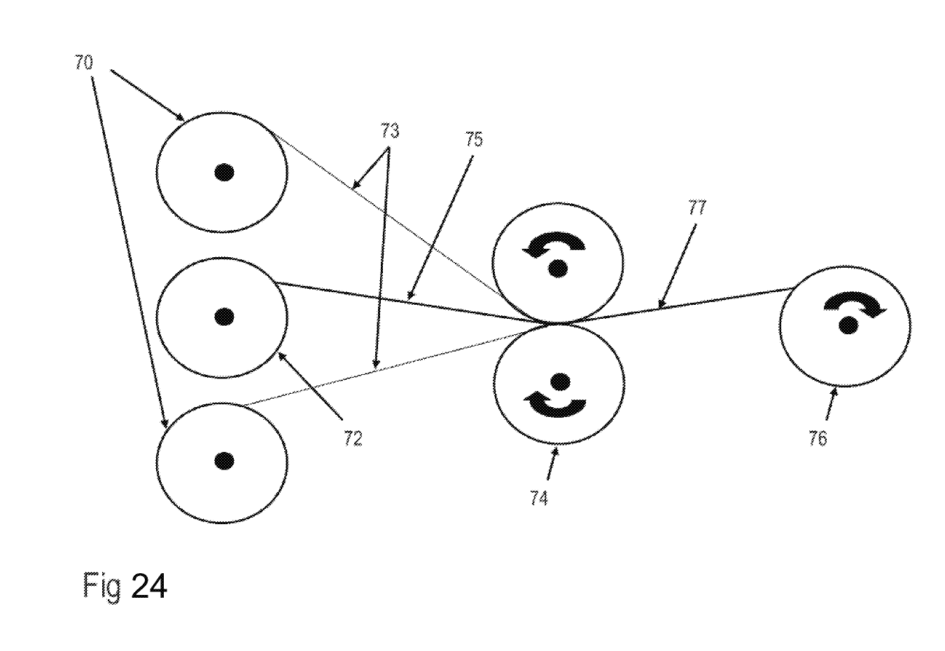

In a second embodiment, shown in FIG. 23, there are two sets of opposing fixed brackets 24 and adjustable brackets 56, 58, one set positioned above the other in two layers, producing in effect a cross-hatched pattern. This embodiment would employ a single outer frame to which both sets of brackets are attached, so that the two sets of brackets are locked in orthogonal alignment with respect to the location of the x-ray focus. The advantage of this embodiment is that the scatter rejection would be considerably improved when compared to a conventional linear grid or that of the first embodiment, because scatter deflected into planes parallel to the grid ribbons in the first layer would be rejected by the transversely directed ribbons in the second layer.

In both embodiments the resulting grid is preferably covered on both top and bottom surfaces with thin Mylar polyester sheets to prevent entry of foreign materials, such as dust, into the open channel spaces, that might cause image shadows. Mylar film is substantially transparent to x-rays.

In another potentially less costly embodiment, the foil ribbons are made of lead or bismuth foil, possibly mixed in alloys also containing tin, antimony, indium or cadmium. In at least some of these cases, the resulting foil will not have sufficient tensile strength to be held in tension on the frame 24, and will require reinforcement with, e.g., a thin layer of Mylar or Kapton tape, such as on one or both surfaces of the foil. The films of Mylar or Kapton used for such tapes are usually about 1 mil in thickness. Alternatively a single steel reinforcing ribbon of the same thickness can be used. Alternatively, the heavy metal can be plated on high tensile strength steel or stainless steel foils rather than Mylar. The steel foil need only be 20-50 microns thick.

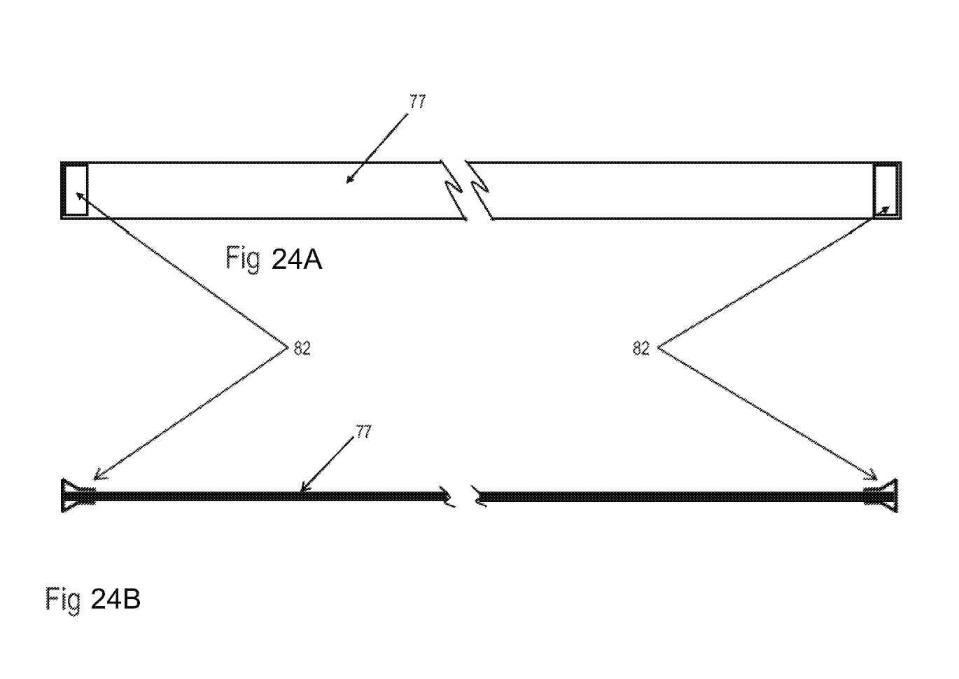

A preferred process for assembly of this reinforced foil is shown in FIG. 24, where two rolls of Mylar or Kapton tape 70 are adhered to the opposite major surfaces of the lead, or other heavy metal foil or alloy foil 72, then fed through compression/gauging rollers 74 then onto a take-up reel 76. A similar process may be used with tapes constructed of polymer loaded metal powders of suitable heavy metals.

In the embodiment employing reinforced lead or alloy foils, it will not be possible to fold the ends of the ribbons creating a similar stop to fix the ribbon in position and to align it with the centerline of the bracket slit. Referring now to FIG. 24B, the reinforced foil ribbons 77 are cut to precise lengths as required to traverse the frame but a steel, brass or other suitable metal clip is formed to a triangular shape in cross-section 82 and crimped over the ends of the reinforced ribbon. In this fashion, the crimped metal clip 82 forms a similar terminus on the reinforced foil ribbons so as to fix the position in the bracket slits and to align the foil to the slit margins.

In an example of this embodiment, a grid is prepared to reduce the scatter radiation for image receptors up to 43 cm×43 cm in size, where the x-ray source 10 focus to the image receptor 14 is a distance of 100 cm. The grid ribbons are constructed of tungsten foils 10 mm high (“L”) and 100 microns in thickness, and cut to a length of 44 cm. A length of 4 mm at each end is folded to produce the triangular stop 34. Brackets 44 are produced with slits 28 cut by wire electrical discharge machining (wire EDM) or laser cutting, to provide a slit width of 150 microns. The slits 28 would be spaced along the brackets with an angular alignment between center planes of 0.0573 degrees with respect to the x-ray focus and a depth of 10.5 mm. The brackets can be constructed of angle steel beams with L-shaped cross-sections with a thickness of 3 mm and web diameters of 11 mm. The grid frame is constructed of mild steel alloy with an inner open area of 45 cm×45 cm with a thickness of 3 mm and a depth of 15 mm. The heavy metal ribbons preferably should be arranged so as to be separated by a distance of about 1 mm.

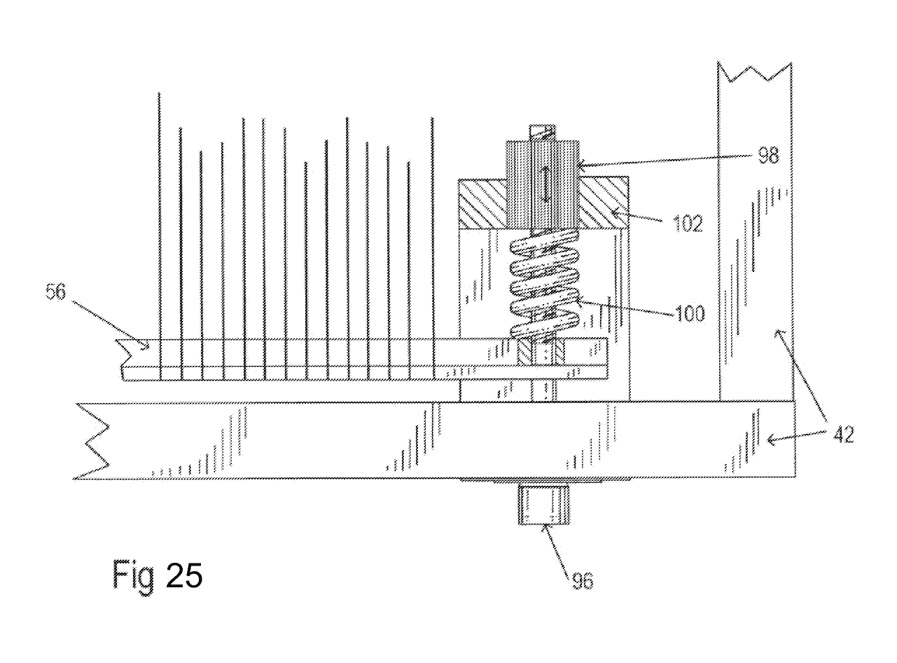

One embodiment of the tensioning mechanism for the adjustment of the movable frame bracket 56, as shown in FIG. 25, is provided preferably at both ends of the movable bracket 56. The tensioning mechanism is constructed using an m6 bolt 96, a coil spring 100 at each end of the adjustable ribbon bracket 56. The top and bottom surfaces of the grid frame would be covered with 25 micron Mylar sheets held in tension to prevent foreign material that might cause image artifacts from entering the space between ribbons.

In a further preferred example of this embodiment the outer grid frame of the first example is increased in depth to accommodate a second layer; The second layer contains a second set of brackets and ribbons essentially identical to that in the first layer, except that the slit separation angle would be increased to 0.0579 degrees with respect to the x-ray tube focus.

Continuing with the description of FIG. 16, a uniform beam 18 of x-rays is directed toward a surface of the object 200 and travels through the object 20 emerging from an opposite surface of the object 20. Differences in x-ray attenuation along linear paths through the object 200 produce an x-ray intensity pattern that comprises image information recorded by the image receptor 14. The image receptor 14 can be a device such as an intensifying screen coupled with a photographic film or any layer of x-ray sensitive material or x-ray sensitive electronic medium, which through one or more steps converts the x-ray intensity pattern into a visible image or visible format.

The x-ray-transparent solid materials, forming the main body of the grids of this invention are preferably formed of a rigid polymer composed mainly of relatively low atomic number elements, (e.g., Hydrogen and Carbon, and possibly Oxygen, and Nitrogen) and have a physical density preferably less than 1.2 g/cm³ and are thus substantially transparent to x-rays. The x-ray transparency of these materials can be further enhanced by adding a foaming agent or micro bubbles to the polymer formulation during the molding process to further reduce the density, and thus increase the transparency of the final material.

The unscattered and focused beams may or may not have been attenuated when passing through the object being examined. X-rays which were scattered during the passage through the object being examined, do not follow a focused path through the channels of the focused grid, and thus intersect and impinge upon the x-ray absorbent boundaries of the focused channels and are thus absorbed by these layers. The absorbent layers are preferably formed of heavy metals such as Lead, Bismuth, Tungsten, or Tantalum. The layers of metal can also be made from low melting point alloys such as Low 117, Low 251 and Low 281, which are alloys of Bismuth with various combinations of Lead, Strontium, Cadmium and Indium. The layers of metal may not necessarily be pure metal, but may contain binding agents (e.g., polymers) to bind a relatively high concentration of heavy metal in the form of a fine powder or as a compound mainly containing elements with atomic numbers greater than 40. Depending on the application for which the antiscatter grid is being used, the relatively high concentration of heavy metals may be in the range of 40% to 98% by weight or volume of the absorbent layer.

Currently available grids are typically specified in terms of grid ratio, i.e., the ratio of channel depth to channel diameter or width. The same approach can be used for the focused grid of the present invention where the grid ratio is (L/W) (i.e., the ratio of channel depth, L, to channel width, W). A desirable set of dimensions for a grid—particularly a grid used generally for radiography purposes—is that the channel width, i.e., W, is approximately 1 mm. Thus, for a highly preferred range of grid ratios of 8:1 to 16:1, the channel depth will fall in the range of 8-16 mm.

An important performance characteristic of a grid is called the primary transmission P, which is defined by the following formula:

P=s/(s+t)e^{-μ(E)L}

where t is the metal layer thickness as shown in FIG. 5, and it is assumed that W=s√2. The second term of the equation, viz., (e-μEL), is an expression that reflects the attenuation of the x-rays as they pass through the focused grid of the present invention, where L is the depth of the focus grid and μE is a linear attenuation coefficient for x-ray photons, of energy E, in the x-ray transparent material from which the arc-shaped modules are made.

The primary transmission P represents the percentage of transmission that passes through the x-ray transparent material for a certain width, W, and depth, L, of the material and metal layer thickness, t. For a channel width, W, of 1.414 mm, the metal layer thickness would range from 0.0525 to 0.25 mm for primary transmissions, P, that range from 95% to 80% without the x-ray transparent material, respectively. For a channel made with a polymer material, the attenuation coefficient, μE, will vary with x-ray energy and with polymer density, which desirably should be less than 1.2 g/cm3. Considering the geometry and attenuation of the polymer material, the total primary transmission at 50 keV will range between 61% and 72% depending on metal thickness, t, and the density of the polymer material.

The various aspects, characteristics and architecture of the device and method of the present invention have been described in terms of the embodiments described herein. It will be readily understood that the embodiments disclosed herein do not at all limit the scope of the present invention. One of ordinary skill in the art to which this invention belongs can, after having read the disclosure, may readily implement the device and method of the present invention using other implementations that are different from those disclosed herein but which are well within the scope of the claimed invention, as defined by the following claims.

What is claimed is:

1. A focused grid for eliminating scattered x-rays while passing with minimal loss primary x-rays moving directly from the x-ray source, the grid comprising:
a plurality of equally spaced strips formed from radiation-absorbent materials, where the major surfaces of the strips extend substantially parallel to the direction of the primary x-rays and the upper edges of the strips are substantially parallel one to the other;
a low molecular weight rigid, foamed material filling the spaces between the spaced strips, the foamed material being highly translucent to the primary x-rays;
the strips being of equal thickness and are so juxtaposed one to the other that all are separated by an equal distance, so as to focus on the line of origin of the primary x-rays, the centrally located strips having major surfaces extending substantially perpendicularly to the focal plane of the x-rays;
rigid edge frames surrounding the grid and top and bottom covers of x-ray translucent material over the grid;
whereby the foam is sufficiently rigid so as to be able to hold the strips in alignment without other tensioning means.

2. The focused anti-scatter grid according to claim 1, wherein the strips are each inclined relative to the adjacent strips so as to converge to a single focus line in a plane parallel to the surface of the frame that contains the locus of an x-ray source; the strips at one edge of the grid being inclined at an opposite angle to the strips at the opposite edge of the grid, and the central strips being substantially perpendicular.

3. The focused anti-scatter grid according to claim 2 wherein the inclined angle of the strips reverse relative to a central line of the anti-scatter grid, along the focus of the x-radiation, so that the incline is reversed on the two sides of the central line.

4. The focused anti-scatter grid according to claim 4, wherein the ratio of the height of the strips to the spacing between the strips is in the range of from 3:1 to 20:1.

5. The focused anti-scatter grid according to claim 1, wherein the radiation-absorbent material forming the strips are a heavy metal selected from the group consisting of lead, bismuth, tungsten, tantalum or alloys thereof, and which have a thickness of between 10 microns and 1000 microns.

6. The focused anti-scatter grid according to claim 1, wherein the strips in a grid set are of equal length, and are pre-tensioned and held in tension by the foam, to maintain the planarity of their outer surfaces and straightness and alignment to the focus of the x-radiation.

7. The focused anti-scatter grid according to claim 1, wherein the upper end of all of the strips in a grid set are all parallel and in the same plane, and are pre-tensioned and held in tension by the foam, to maintain the planarity of their outer surfaces and straightness and alignment to the focus of the x-radiation.

8. An improved focused, anti-scatter grid, comprising a frame, and two transversely aligned anti-scatter grids according to claim 1, so as to form a combined crossed, linear focused grid, both grids being so arrayed as to have focus lines which lie in the same plane, with the convergence at the location of the x-ray source.

9. The focused grid of claim 4, where the spacing between the strips is in the range of from 0.2 mm to 1 cm.

10. The focused grid of claim 5 where the heavy metal alloy is a low melting alloy combining Bismuth and at least one of Lead, Strontium, Cadmium and Indium.

11. The focused grid of claim 1 where the polymer material is a substantially rigid, foamed polymer composed mainly of the low atomic number elements Hydrogen, Carbon, Oxygen or Nitrogen, and having a density of less than 1.2 grams per cubic centimeter, so as to be substantially radiation-transparent.

12. The focused grid of claim 10 where the rigid polymer is selected from ABS, Urethane, foamable PVC, polyimide, or acrylic polymers.

13. A method for manufacturing a focused grid for eliminating scattered x-rays while passing with minimal loss primary x-rays moving directly from the x-ray source, the grid comprising a plurality of equally spaced strips formed from radiation-absorbent materials, and a low molecular weight rigid foamed material, formed of low atomic weight atoms, filling the spaces between the spaced strips, the foamed material being highly translucent to the primary x-rays; the method for manufacturing comprising:
providing a framework comprising a bottom surface and two pairs of parallel rigid frame edges fully enclosing an inner volume, one pair of frame edges including a support surface on each edge supporting a series of pins extending outwardly from the support surface, the two series of pins being so juxtaposed that opposing pins are substantially parallel to each other and the pins on either side of a central portion of the support surfaces each being slightly more inclined relative to the adjacent pins beginning from the central pins, the pins being of substantially equal thickness and are so juxtaposed one to the others as to focus on the line located a predetermined distance above the support surfaces, the centrally located pins extending substantially perpendicularly from the support surfaces;
one of the pin-supporting frame edges being movable longitudinally with respect to the other frame edge;
forming identical loops of the radiation absorbent strips and having the loops extending around the opposed sets pins such that a loop extends from a pin on the movable pin-supporting frame edge to a pin on the other pin-supporting frame edge, which are parallel one to the other and located in the same plane;
moving the pin-supporting frame edge longitudinally away from the other pin-supporting frame edge until a predetermined tension is achieved on the foil loop so as to align the foil loop surfaces such that the major surfaces are straight and the upper edges of the foil loops are parallel each to the other;
sealably enclosing the tensioned loops and pins and frame edges, forming a sealed space including the loops and pins; and
feeding a foamable polymer into the sealed space and causing the foamable polymer to foam and set to form a rigid foamed polymer firmly holding the tensioned loops in the preset position;
whereby the foam is sufficiently rigid to maintain the loops in the alignment when the tension from the movable pins is removed.

14. The method for manufacturing the focused grid in accordance with claim 13, further comprising separating the rigidly held tensioned loops from the pin-supporting frames after the rigid foamed polymer has set, leaving the foil loop surfaces held in position by the set foamed polymer.

15. The method for manufacturing the focused grid in accordance with claim 13, wherein the heavy metal foil is attached to and supported by a thin polymer film.

16. The method for manufacturing the focused grid in accordance with claim 13, wherein the foamable polymer is composed mainly of the low atomic number elements Hydrogen, Carbon, Oxygen or Nitrogen, and has a density of less than 1.2 grams per cubic centimeter when foamed, so as to be substantially radiation-transparent when foamed.

17. The method for manufacturing the focused grid in accordance with claim 13, wherein the foil loops are formed from a heavy metal selected from the group consisting of lead, bismuth, tungsten, tantalum or heavy metal alloys thereof, and which have a thickness of between 10 microns and 1000 microns.

18. The method for manufacturing the focused grid in accordance with claim 13, where the heavy metal alloy is a low melting alloy combining Bismuth and at least one of Lead, Strontium, Cadmium and Indium.

19. A focused grid comprising: a plurality of modules, each module comprising alternating radiation transparent materials and radiation-absorbent materials, the modules being so assembled such that adjacently positioned modules are so juxtaposed one with each other to form focused channels, and a frame onto which the plurality of modules are mounted and secured in the desired juxtaposition.

20. The focused grid of claim 19 where the modules are substantially identical to each other and comprise thin, heavy metal ribbons, wherein the frame supports the plurality of thin, heavy metal ribbons so separated and juxtaposed as to define channels through the air space defined by and between the ribbons, the channels focusing any radiation passing through them along a single focus line, the focus line contains the locus of an x-ray source, the channels comprising primarily only air.

21. The focused anti-scatter grid according to claim 20 wherein the frame comprises at least two opposed brackets that each incorporate a plurality of holders to support the heavy metal ribbons in planes orthogonal to the long axis of the brackets, and that are each inclined relative to each other so as to converge to a single focus line in a plane parallel to the surface of the frame that contains the locus of an x-ray source.

22. The focused anti-scatter grid according to claim 21, wherein each of the brackets define a slit for holding the ribbons at the inclined angle relative to each other, and the planes of the ribbons are arrayed so as to describe a fixed angle from the focus line, with respect to the planes bisecting adjacent slits in which the adjacent ribbons are held.

23. The focused anti-scatter grid according to claim 22, wherein the ratio of the depth of the ribbons to the spacing between the ribbons is in the range of from 3:1 to 20:1.

24. The focused anti-scatter grid according to claim 21, wherein the thin, heavy metal ribbons are formed from a heavy metal selected from the group consisting of lead, bismuth, tungsten, tantalum or alloys thereof, and which have a thickness of between 10 microns and 1000 microns.

25. The focused anti-scatter grid according to claim 24, wherein the ribbons in a grid set are of equal length, and are held in tension by the frame, to maintain their straightness and alignment to the focus.

26. The focused grid of claim 25 comprising a frame which incorporates two transversely aligned sets of ribbons so as to form a crossed linear focused grid, both sets of ribbons being so arrayed as to have focus lines which lie in the same plane, with the convergence at the location of the x-ray source.

27. The focused grid of claim 20 where the spacing between the ribbons is 0.2 mm to 1 cm.

28. The focused anti-scatter grid according to claim 1 further comprising:
i. an open rectangular frame containing two brackets on two opposing sides of the grid, each bracket incorporating a plurality of narrow slits where the planes of the slits are orthogonal to the long axis of the brackets and are each inclined in the direction along the slit, to converge to a single line (focus line) in a plane parallel to the surface of the frame that contains the locus of the x-ray source;
ii. the planes of the slits are not parallel but are arrayed along the brackets so that the plane bisecting the slit describes a fixed angle from the focus line, with respect to the planes bisecting adjacent slits; the planes of the slits on one bracket are precisely aligned with the slits on the opposing bracket;
iii. the depths of the slits in the brackets along the planes extending to the focus line are sufficient so that the ratio of the depth to the spacing between slits is between 3:1 and 20:1;
iv. thin, heavy metal ribbons extend through the slits in each bracket to the opposing bracket, and have a width corresponding to the depth of slits, and thickness in the range of from 10 microns and 1000 microns;
v. the width of the slits is formed to exceed the thickness of the ribbons by 50-100%; and
vi. the two brackets extend substantially parallel to each other so that all ribbons in a grid are of equal length, sufficient to exceed the spacing between corresponding brackets across the open frame.

29. The focused anti-scatter grid according to claim 28 wherein the brackets are constructed in pairs where one bracket is fixed to the outer open frame and the opposing bracket is loaded under spring tension to ensure that the ribbons between them are aligned to the focus.

30. A focused grid of claim 28, further comprising a frame comprising two pairs of vertically arranged brackets forming a crossed linear grid, the upper pair of brackets orthogonal to the lower pair; the ribbons extending between both pairs of brackets converge to orthogonal focus lines which lie in the same plane with the convergence at the location of the x-ray source.

31. The focused grid of claim 28 where the metal foil ribbons are constructed of copper, steel or alloys thereof with an over-coating of tin or antimony for use in mammographic applications.

32. The focused grid of claim 28 where the heavy metal foil ribbons are made of a material with poor tensile strength, selected from the group consisting of lead, tin, antimony bismuth and alloys thereof and further comprises a surface coating or adhered layers of a high tensile strength polymer, where the foil ribbon is strengthened on one or both surfaces of the ribbon; each ribbon having an opening at each end to hold a rigid rod to provide fixation and centering to the ribbons under tension.

33. The focused grid of claim 30 wherein the length of the brackets and the distance between the brackets can be in the range of three centimeters to 4 meters, and depends upon the application intended.

34. The focused grid of claim 31 where the bracket slits are machined to achieve focus distance ranging from a few tens of cm to 2 m or more.

35. The focused grid of claim 31 where the spacing between bracket slits is in the range of from two tenths of a mm to at least 5 cm, depending on the application required.

36. The focused grid of claim 31 where the bracket slits are machined to locate the focus point along an orthogonal to the surface of the grid frame through the center of the grid frame opening.

37. The focused grid of claim 34 where the bracket slits are machined to locate the focus point along an orthogonal to the surface of the grid frame that is through the midpoint of one inner edge of the grid frame.
